(19) United States
(12) Patent Application Publication
(10) Pub. No.: US 2008/0131562 A1
(43) Pub. Date: Jun. 5, 2008

(54) Method for the Preparation of Tea Beverages Made of Black or Green Tea

(75) Inventors: Helmut Korber, Halle (DE); Friedemann Pieschel, Bad Soden (DE)

Correspondence Address: LERNER GREENBERG STEMER LLP, P O BOX 2480, HOLLYWOOD, FL, 33022-2480, US

(73) Assignee: Helmut Körber, Halle (DE)

(21) Appl. No.: 11/815,218

(22) PCT Filed: Jan. 31, 2006
(86) PCT No.: PCT/EP06/00840

§ 371 Date: Aug. 1, 2007

(30) Foreign Application Priority Data
Feb. 1, 2005 (DE) DE102005004815.3

(51) Int. Cl. A23F 3/00; G01N 33/14; B65B 29/02; A47J 31/08

(52) U.S. Cl. 426/78; 426/597; 426/231; 99/279

(57) ABSTRACT

A method of preparing hot tea for immediate use uses green or black tea leaves or mixtures of the leaves and untreated water that originates from the local supply network as the brewing water. The novel method aims to prevent clouding and the formation of a skin after the brewing process with little effort, cost-effectively and without influencing the flavor or hygienic properties of the tea. Prior to or during the heating of the brewing water, or during the brewing process, potassium-, sodium- or ammonium citrate are added individually or as a mixture, to produce a pH value of between 5 and 7 in a purely aqueous solution. As a result of the addition, the brewed tea is translucent and devoid of the usual clouding and formation of a shimmering skin on the surface.

The invention relates to a procedure for the preparation of tea beverages by using tea leaves of black or green tea or mixtures of these and boiling water for the improvement of the quality of the infusion.

It is well known that the quality of infusions of black or green tea does not only depend on the quality of the tea leaves used and on their processing but also on the quality of the boiling water used to a very decisive degree. In this context, the issue is not defects in the quality of the water used such as the own taste of the water/odour or even harmful ingredients but about totally harmless and omnipresent hardness producing substances which even increase the value of the water from a health perspective and which must be in the water in a minimum quantity in order to protect the local water pipework. For example, the German drinking water ordinance prescribes an acidity of at least 1.5 mol/m³ which corresponds to a carbonate hardness of approx. 4°dh.

Usually, the consumer has a supply of drinking water at his/her disposal which has a considerably higher carbonate hardness. However, the use of such types of water as boiling water for brewing tea entails a number of disadvantages.

A beverage brewed from black tea contains polyphenolic compounds which are relevant for the desirable healthy, organoleptic and aesthetic attributes of the brewed beverage to a high degree. If water which has a carbonate hardness of more than 4 degrees of German hardness is used for brewing tea, black tea will get cloudy only a short time after the beverage is brewed and an easily visible surface film usually forms.

The surface film which has formed in this way frequently sticks to the wall on the inside of the cup and makes cleaning of the cups harder. In addition, the colour becomes darker and darker and turns from a dark red-brown to black, which frequently even looks grayish-black on account of the cloudiness of the drink. These aspects have a detrimental effect on the aesthetic attributes of the beverage.

It is known that the water can be boiled several times or boiled for a relatively long period of time in order to eliminate this disadvantage, during which process the hydrogen carbonates of the hardness-producing substances decompose, carbon dioxide is released and lime is separated.

Moreover, filter devices which eliminate the hardness-producing substances by means of an ion exchange process are also known. However, these can only be made available after preparation (time for swelling) or under consideration of precautions (cooled storage against growth of bacteria) and they need special filter containers. In practical use, this method creates an increased effort and additional costs.

EP 0481 262 B1 describes a method, in which the polyphenolic compounds, which are contained in moist tea leaves, are oxidized by heating them at a pressure exceeding the water vapour pressure at reaction temperature. The tea leaves treated in this way are processed into instant tea products.

However, the beneficial health effects of the polyphenolic compounds are lost on account of the oxidation.

The invention is based on the task of proposing suitable measures which prevent a “cloudiness” of the beverage and the formation of a surface film on it after the process of brewing black or green tea, which require a low effort, which are cost-effective and which do not influence the flavour and health properties of the tea beverage.

According to this invention, the task is resolved by means of the characteristics specified in claim 1. Advantageous designs and further developments constitute the subject of claims 2 to 10. Claims 11 to 13 refer to special uses, whereas the appertaining tea bags, tea filters or the appertaining carrier material constitute the subject of claims 14 to 17.

The result that a cloudiness of the tea or the formation of a surface film was prevented entirely after brewing of the tea by adding potassium, sodium or ammonium citrates either individually or as a mixture which resulted in a pH-value of 5 to 7 in a purely aqueous solution either before or during heating of the water for brewing the tea or during the brewing process as such was a total surprise.

Water for brewing tea is defined as water coming from the local water supply grid which was not submitted to further treatment before brewing the tea.

The tea brewed was still entirely transparent and without the formation of a shimmering film on the surface, which is common otherwise, after it had cooled down entirely. Even after the tea had been left for several hours, no change in the pattern of properties could be discerned. The overall aesthetic impression of the tea beverage was excellent.

With the help of experiments it was ascertained that the addition of citrates and mixtures thereof at concentrations of up to approx. 2 g of citrate per l in distilled water as per this invention is perceived as neutral in taste even though there is a pH value of approx. 6. This means the proposed citrates and/or citrate mixtures can be used in concentrations of up to 2 g of citrate per l of brewing water.

In case the water used only has the minimum carbonate hardness specified by law (approx. 4°dH), a pH value of 6 is not undercut and, hence, an acidification effect is not perceived. In case water with a carbonate hardness of 25°dH is used for preparing the tea beverage, which happens only very rarely in practice, the acid capacity of the citrate addition is not sufficient to destroy all bicarbonates any more. However, regardless of this, the precipitation of Mg hydroxocarbonates and calcium carbonate is prevented in those cases. By means of the combination of the slightly acidifying and good complexing effects of the citrates according to the invention, an acidic reaction of tea infusions which can be perceived in the taste becomes impossible, even though there is no unwanted cloudiness in the tea beverage on account of carbonate precipitation.

The proposed citrates and their mixtures are sufficiently strongly complexing in order to avoid any precipitation of hardness-producing substances at boiling temperature. At the same time, they also complex non-toxic heavy metal traces contained in the potable water to such a degree that the precipitations and the discolouration in the tea infusion caused by these are slowed down considerably.

It appears that the quality of the brewing water was improved to such a degree by means of the addition of citrate that the polyphenols, which are beneficial to human health, were not transformed into insoluble compounds and that, hence, they can develop their health-promoting effect as anti-oxidants within the human body after the tea has been consumed. Moreover, it is also advantageous that the pH value of the water did not change materially during the brewing process on account of the addition of citrate. It is generally known that there are slightly alkaline reactions which have a detrimental effect on taste and odour in case drinking water with a high carbonate hardness is used during brewing.

In particular diammonium, dipotassium and disodium citrate can be used as citrates in a mixture with up to 90 mol % of monoammonium, monopotassium and monosodium citrate. The quantities to be used are very low and amount to at least 0.1 g of citrate per l of brewing water. Preferably, the upper limit amounts to 2 g of citrate per l of brewing water. Physiologically, the ingestion of citrate associated with drinking the tea beverage is absolutely harmless.

It is known that citrates play a central role in the human body (carbohydrate metabolism (“citric acid cycle”)). Moreover, the foodstuff additive ordinance does not specify any upper limit for the addition of citric acid and, hence, of citrate to foods.

In the framework of a series of experiments, it has been possible to ascertain that the quantities of citrate can be reduced down to the lower limit specified with a reducing carbonate hardness of the brewing water. In case of a medium carbonate hardness, very good results were achieved with citrate additions which were as low as 0.2 g per l of brewing water.

The citrates used are products which are commercially available. They can also be made by means of the neutralization of citric acid with the appropriate stoichiometric amount of soda, potassium, ammonia water and so on or by mixing citric acid with triammonium, tripotassium or trisodium citrate in a molar relationship of approx. 1.9:1.1 to 1:2. In case citric acid is mixed with its tertiary salts, a solution and isolation of the solid salts can be dispensed with in case of a use in the solid state. The pH value, which the citrates and their mixtures result in in a solution of approx. 0.1 percent, should preferably amount to between 6 to 6.5 and should not be perceived as acidic as regards its taste.

Compared with the other known measures, the low quantities of citrate used only lead to very low additional costs. As an alternative, the required quantity of citrate can either be added to the brewing water or to the tea to be brewed. The step of adding the citrate directly during the brewing process can be prevented by adding the required quantity of citrate as early as during the fabrication of the tea bags in the case of the quite common use of tea bags filled with tea leaves.

There is also the possibility of soaking the material for the tea bags with a citrate solution. Using an analogous process, it is also possible to soak carrier materials such as strips of paper in a citrate solution which is then inserted into the brewing water immediately before the brewing process, with the citrate being dissolved by bringing it into contact with the brewing water. In case disposable tea filters are used, there is the possibility of soaking these with a citrate solution as early as during the production process. In the course of the contact with the brewing water, the citrate is then dissolved from the filter. Hence, the required devices for the preparation of green or black tea, such as tea bags, tea filters or carrier materials, which are directly connected with the implementation of the idea of the invention, also constitute the subject of the invention filed.

The invention also relates to the use of potassium, sodium or ammonium citrates, which are known as such, individually or as a mixture, as an additive to improve the quality of the brewing water for the preparation of tea beverages by means of the use of tea leaves of black or green tea or mixtures of these. The use of citrates is restricted to those citrates which result in a pH value of 5 to 7 in a pure aqueous solution. In this case, “mixtures” are defined as the commercially available packages offered by the manufacturer.

The citrations achieved by means of the addition of citrate as per this invention are neutral in taste just like ammonium, sodium and potassium ions so that the taste of the tea brewed is not affected.

As an additional advantage it turned out that, in case chlorinated water is used as brewing water, the addition of a citrate additive before heating up the brewing water prevents the formation of an unsavoury flavour which is caused by the influence of chlorine on the tea. On account of this, the use of filters containing activated carbon in order to avoid the flavour from being affected by chlorine can be dispensed with.

The surprising effect of the potassium, sodium and ammonium citrates added is not solely attributable to their buffer effect, which is known as such, but only in combination with the formation of complexes which cannot be foreseen.

Comparative experiments during the process of brewing black tea by adding solutions with potassium phosphates, tartrates, lactates and acetates, which were brought to comparable molar concentrations and to comparable pH values (5 to 7) by adding potash or free acid, still resulted in “cloudiness” and the formation of an undesirable “surface film”.

### EXAMPLES

### A: Experiments without the Addition of Citrates

### Experiment A1:

1 l of drinking water (hard) from a regional water supply grid (Bad Soden) with a total hardness of 24°dH and a carbonate hardness of 18°dH was heated in an electric kettle until said kettle switched off automatically once the water was boiling strongly. A commercially available tea infusion bag with “black tea” of a brand of tea sensitive to hardness was placed in a 400 ml glass cup with a high format and, afterwards, 200 ml of the brewing water which had been prepared was filled in (still boiling).

After 1 minute after filling in the brewing water, the tea bag was lifted to above the surface of the water and lowered again. This process was repeated twice at intervals of one minute each. After another minute, the teabag was lifted out of the brewing water and allowed to drain properly; however, it was not squeezed out. This means the entire brewing period for the tea beverage T1 amounted to 3 minutes.

### Experiment A2:

Using a method analogous to the one described under A1, a further experiment was carried out in parallel using soft drinking water (originating from a municipality within the Taunus region) with a total hardness corresponding to a carbonate hardness of 4°dH and the tea beverage T2 was obtained after a total brewing time of 3 minutes.

### Experiment A3:

This experiment was carried out analogously to experiment A1 with “green tea” sensitive to hardness in the form of tea leaves with a quantity of 2 g used instead of “black tea”. The mixture was stirred with a tea spoon left in the glass at the minute intervals specified and the tea liquid was poured into a second glass cup as tea beverage T3 after the end of the brewing time (3 minutes).

### Experiment A4:

One further experiment with drinking water (soft) with a total hardness corresponding to a carbonate hardness of 4°dH was carried out in parallel to this by using a method analogous to the one described under A3 and tea beverage T4 was obtained in this way after a total brewing time of 3 minutes.

After intervals of 1, 3, 10 and 30 minutes as well as of 1 hour and 24 hours after taking out the tea bag, the tea beverages T1 and T2 as well as the tea beverages T3 and T4 were inspected visually with regard to cloudiness, the formation of a surface film and darkening of the colour.

### Experiment A5:

A tea beverage was prepared in a similar manner as described under A1; though in this case chlorinated, medium-hard drinking water from the town of Grafenwöhr (total hardness 12°dH, carbonate hardness 10°dH) was used in deviation to experiment A1.

The tea beverage obtained in this way was clearly cloudy and displayed a stronger formation of a surface film. Moreover, this tea beverage had an unpleasant aftertaste.

### Evaluation of Tea Beverages T1 and T2:

Tea beverage T1 already displayed a slight cloudiness with an easily visible surface film as early as at the end of the brewing time.

After 10 minutes, the surface film of tea beverage T1 had become “firm”, the tea was clearly cloudy and had visible darkened in colour. Moreover, tea T1 had a fishy aftertaste and odour. After one hour, the tea had become entirely black.

In contrast to this, tea beverage T2 remained almost unchanged and displayed a pleasant dark-brown colour which was preserved even after 24 hours. There were no visual indications of cloudiness or of the formation of a surface film. Within a period of time of 24 hours only a slight darkening of the colour was found.

### Evaluation of Tea Beverages T3 and T4:

Though brewing with boiling water is not considered the optimum way of preparing green tea as regards its taste, it constitutes a more stringent version of the test as regards cloudiness and the formation of a surface film compared with brewing at lower temperatures.

On tea beverage T3, the formation of a surface film and a slight cloudiness were already discernible after pouring it into the second cup.

Unlike this, tea beverage T4 remained almost unchanged.

A considerable darkening of the colour comparable to the one witnessed in the case of the black tea was not observed in the tea beverages T3 and T4 even after a period of 24 hours.

### B: Examples According to the Inventions (with Added Citrate)

### Example 1

“Black tea” was brewed in a manner similar to the method employed in example 1; in this case, 1.2 ml of a dipotassium citrate solution (1.2 ml of this solution weigh 1.4 g and it contains 0.84 g of solid dipotassium citrate) was added to the brewing water (1 l) before the kettle was put into operation.

This was obtained by adding an almost saturated potash solution to the stoichiometric quantity of crystalline citric acid (monohydrate) in steps, which resulted in an almost saturated viscous solution of dipotassium citrate. In a separate test, 3.3 g of this solution were mixed with 1 l of distilled water and resulted in a pH value of 6.1. In a test of the flavour carried out by several persons, this could not be differentiated from pure distilled water.

The tea beverage obtained displayed a pleasant dark brown colour which only darkened slightly after 30 minutes. As regards the taste no differences were found in comparison with tea beverage T2.

Even after a period of 24 hours both the formation of a surface film and cloudiness could not be discerned, even though the tea had reached a clearly darker colour.

### Example 2

A commercially available strip (5 cm×10 cm) of filter paper was soaked with 1.2 ml of dipotassium citrate solution according to example 1 and dried.

Using the same method as in experiment T1, “black tea” was prepared, during which process the strip of paper soaked in citrate solution was inserted into the brewing water (1 l) before the kettle was put into operation.

The tea beverage obtained in this way corresponded to the tea beverage according to example 1 as regards the flavour and the visual impression.

### Example 3

5 drops of dipotassium-citrate solution (0.3 g) (corresponding to 0.18 g of solid dipotassium citrate) were applied on a commercially available tea infusion bag for “black tea” in accordance with example 1 and this was used for preparing tea using the same method as described in experiment A1 after air-drying of the tea bag.

The tea beverage obtained corresponded to the tea beverage according to example 1 as regards the flavour and the visual impression.

### Example 4

“Black tea” was prepared using the same method as the one described in example 1 with the difference that water of a different quality was used, i.e. chlorinated medium-hard drinking water from the town of Grafenwöhr (total hardness 12°dH, carbonate hardness 10°dH).

The tea beverage obtained corresponded to the tea beverage according to example 1 as regards the flavour and the visual impression.

### Example 5

A tea was prepared with chlorinated drinking water according to example 4 using the method described in example 3. The colour and the appearance of this tea beverage corresponded to the tea beverage obtained under example 4 at all times. However, the same unpleasant odour and aftertaste as in the case of the brewing experiment without the addition of citrate quickly emerged (experiment A5).

The comparison of example 4 with example 5 shows that the destruction of chlorine requires the previous boiling process with citrate in order to make sure that the unpleasant aftertaste does not develop upon the contact with the tea.

### Example 6

2 g of “green tea” were mixed with 0.71 g of finely ground, commercially available diammonium citrate and brewed into a tea beverage using a method similar to the one employed for experiment A3, however, by using a different water quality, i.e. medium-hard water (water from the supply network of the city of Berlin, total hardness 16°dH, carbonate hardness 10°dH).

The tea beverage obtained corresponded to the tea beverage T4 as regards taste and the visual impression.

### Example 7

“Black tea” was prepared using a method similar to the one employed in example 1, the only difference being that 0.17 g of a mixture of 58.8 g of finely ground trisodium citrate dihydrate and 21 g of citric acid monohydrate (both in commercially available qualities) were used as the citrate preparation.

The tea beverage obtained corresponded to the tea beverage according to example 1 as regards the flavour and the visual impression.

### Example 8

“Black tea” was prepared using a method similar to the one employed in example 1, the only difference being that the quantity of the dipotassium solution used was increased to 2.86 ml (2.86 ml correspond to 2.0 g of dipotassium citrate).

Compared with the tea beverage obtained in example 1, the tea beverage obtained displayed a slightly reduced darkening of its colour as early as after one hour and also after a period of 24 hours.

“Black tea” and “green tea” each of the same brand of tea were used in the experiments and examples described herein above.

### Example 9

3 drops of dipotassium-citrate solution according to example 1 were applied on a commercially available disposable tea filter made of material containing cellulose and said filter was dried.

The increase in weight amounted to 0.1 g. The filter was inserted in the mounting designed to that end for brewing the tea. The tea used was 9 g of a commercially available “black tea” of a type which is evaluated as very sensitive to water hardness.

One litre of water of a medium hardness (Wolfen region, total hardness 14°dH, carbonate hardness °dH) was used as the brewing water. After a brewing time of 3 minutes, the desired tea beverage, which was designated 9a, was obtained.

After that, a second brewing process took place with the difference that a citrate-free tea filter was used and a tea beverage designated as 9b was obtained.

Subsequent to that, a third brewing process was carried out with a citrate-free tea filter and drinking water (soft) with a total hardness corresponding to a carbonate hardness of 4°dH. The tea beverage obtained was designated 9c.

Even after a period of one hour, the tea beverage 9a did not display any cloudiness or the formation of any surface film; however, its colour darkened somewhat more strongly than the tea beverage 9c prepared with a citrate-free filter and very soft water.

On the other hand, tea beverage 9b already turned slightly cloudy after 10 minutes and a slight surface film developed. After one hour, the tea beverage 9b was clearly darker and displayed an increased cloudiness. After 24 hours, a sediment had formed on the bottom of the teapot containing tea beverage 9b. In the case of the other two tea beverages 9a and 9b, however, no formation of a sediment was observed.

1-17. (canceled)

18. A process for preparing a hot tea beverage intended for immediate consumption, comprising:
providing tea leaves of black tea or green tea or mixtures thereof;
providing substantially untreated water from a local drinking water supply as brewing water;
adding an amount of a citrate selected from the group consisting of potassium citrate, sodium citrate, and ammonium citrates, having a pH value of 5 to 7 in pure aqueous solutions, separately or as a mixture, before or during a heating of the brewing water or during a brewing process; and
filtering the tea leaves from the brewing water.

19. The process according to claim 18, wherein the citrate is selected from the group consisting of diammonium, dipotassium, or disodium citrate in mixtures with up to 90 mol % of monoammonium, monopotassium or monosodium citrate.

20. The process according to claim 18, which comprises forming a citrate mixture from citric acid and tertiary citrate or by corresponding partial neutralization of citric acid.

21. The process according to claim 18, which comprises adding 0.1 to 2 grams of citrate per liter of brewing water.

22. The process according to claim 18, which comprises adding the amount of citrate to the brewing water.

23. The process according to claim 18, which comprises adding the amount of citrate to the tea leaves or the mixtures thereof.

24. The process according to claim 18, which comprises applying the amount of citrate on a carrier material or soaking material with a citrate solution, and bringing the material into contact with the brewing water.

25. The process according to claim 18, which comprises adding the amount of citrate to a teabag filled with tea or soaking the tea bag with a citrate solution.

26. The process according to claim 18, which comprises applying the amount of citrate on a disposable tea filter or soaking the filter with a citrate solution.

27. The process according to claim 18, which comprises measuring a carbonate hardness of the brewing water and dosing the amount of citrate in dependence on the carbonate hardness of the brewing water, with a reduction of a quantity down to 0.1 grams per liter of brewing water at diminishing degrees of carbonate hardness.

28. A tea additive, comprising:
a citrate selected from the group consisting of potassium citrate, sodium citrate, and ammonium citrate, separately or as a mixture thereof, yielding a pH value of 5 to 7 in a pure aqueous solution, as an additive to tea and/or brewing water for brewing the tea, for preparing a tea beverage for immediate consumption, in combination with tea leaves of black tea or green tea or mixtures thereof and substantially untreated water from a local drinking water supply for brewing water.

29. The tea additive according to claim 28, wherein the citrates are selected from the group consisting of diammonium citrate, dipotassium citrate, disodium citrate, in a mixture with up to 90 mol % of monoammonium citrate, monopotassium citrate, or monosodium citrate.

30. The tea additive according to claim 28, wherein a quantity of citrate amounts to between 0.1 and 2.0 grams of citrate per liter of brewing water.

31. A tea bag for brewing tea, comprising:
a material containing cellulose formed into a bag for brewing tea;
tea leaves of black tea, green tea, or mixtures thereof, contained in said bag; and
an additive of potassium citrate, sodium citrate, or ammonium citrate, separately or as a mixture, which yield a pH-value of 5 to 7 in pure aqueous solutions, said additive amounting to at least 0.1 grams per liter of brewing water.

32. The tea bag according to claim 31, wherein said citrates are added to said bag or said material of said bag is soaked with a citrate solution.

33. A disposable tea filter, comprising a material containing cellulose, for brewing black tea or green tea or mixtures thereof, soaked with a solution based on potassium, sodium or ammonium citrates, separately or as a mixture, which yield a pH value of 5 to 7 in a pure aqueous solution, to an amount of at least 0.1 gram of citrate per liter of brewing water.

34. A carrier material to be brought into contact with water from the local drinking water supply grid, the water forming brewing water for brewing black tea or green tea or mixtures thereof, the material being soaked with a solution based on potassium, sodium or ammonium citrates, separately or as a mixture, which reach a pH value of 5 to 7 in a pure aqueous solution, to an amount of at least 0.1 grams of citrate per liter of brewing water.
